(19) United States
(12) Patent Application Publication
(10) Pub. No.: US 2008/0292726 A1
(43) Pub. Date: Nov. 27, 2008

(54) OPHTHALMIC GALLIUM COMPOSITIONS AND METHODS OF THEIR USE

(76) Inventor: Lawrence R. Bernstein, Menlo Park, CA (US)

Correspondence Address: Lawrence R. Bernstein, 285 Willow Road, Menlo Park, CA, 94025-2711, US

(21) Appl. No.: 12/177,591

(22) Filed: Jul. 22, 2008

Related U.S. Application Data
(63) Continuation-in-part of application No. 12/017,378, filed on Jan. 22, 2008.
(60) Provisional application No. 60/881,920, filed on Jan. 23, 2007.

(51) Int. Cl. A61K 33/24; A61K 31/555; A61K 31/28; A61P 37/00; A61P 31/04; A61P 27/02; A61P 31/12

(52) U.S. Cl. 424/650; 514/184; 514/492

(57) ABSTRACT

Provided are ophthalmic pharmaceutical compositions comprising gallium. These compositions are designed primarily for topical ocular and intraocular administration. Also provided are methods of use for the compositions in the treatment of human and veterinary diseases and disorders. Treatable diseases and disorders include adverse conditions of the eye and adnexal tissues of the eye. These adverse conditions comprise inflammatory conditions, infections, cancers, and other pathological conditions.

### CROSS REFERENCE TO RELATED PATENT APPLICATIONS

This application is a continuation-in-part of application Ser. No. 12/017,378, filed Jan. 22, 2008, which claims the benefit of U.S. Provisional Application No. 60/881,920, filed Jan. 23, 2007. Both of the applications above, to which a benefit is claimed, are herein incorporated by reference in their entireties.

### TECHNICAL FIELD

This invention relates generally to pharmaceutical compositions for ocular administration. In particular, this invention pertains to pharmaceutical gallium compositions for topical ocular or intraocular administration, and methods of their use.

### BACKGROUND OF THE INVENTION

Gallium compounds, including gallium nitrate, gallium sulfate, and gallium maltolate, have been repeatedly shown to have anti-inflammatory, antiproliferative, antimicrobial, and other therapeutic activities when administered systemically (i.e., orally, intravenously, or by other means that introduce gallium into the bloodstream and allow for its distribution through the body). Systemically administered gallium has shown efficacy in animal models of rheumatoid arthritis (U.S. Pat. No. 5,175,006 to Matkovic et al.) as well as in the treatment of cancer and infectious disease (Bernstein LR, PHARMACOLOGICAL REviEws 50:665-682, 1998). Locally administered gallium is effective in treating psoriasis and related dermatologic disorders (U.S. Pat. No. 5,747,482 to Bernstein). Lobanoff et al. (EXPERIMENTAL EYE RESEARCH 65:797-801, 1997) reported that systemically administered gallium nitrate, subcutaneously injected into the neck, was effective in a mouse model of uveitis. Local administration of gallium nitrate to the eye was not contemplated by Lobanoff et al.; furthermore, the use of gallium compounds other than gallium nitrate was not considered, and the treatment of eye disorders other than uveitis was not considered. It has now been discovered that gallium locally administered to the eye can relieve inflammation, inhibit autoimmune and other pathological immune responses, inhibit neoplasia and other pathological hyperproliferative conditions, and treat microbial and viral infections.

Due to the sensitivity of eye to foreign substances, including to many medicinal compounds, and to the poor penetrability of the cornea to many drugs, diseases and disorders of the eye are commonly difficult to treat with locally administered medications. Furthermore, due to the relatively poor blood supply to parts of the eye (such as the vitreous humor, aqueous humor, lens, and cornea), systemic medications commonly do not reach diseased portions of the eye in sufficient amounts to be effective. Common diseases and disorders of the eye that can be difficult to treat include infections (such as bacterial, viral, parasitic, and fungal, of the cornea, iris, uvea, or other parts of the eye), inflammation, autoimmune disorders, cancer, glaucoma, macular degeneration, and retinopathy (including diabetic retinopathy).

Intraocular inflammation, particularly uveitis (inflammation of the uvea, which consists of the iris, choroid, and ciliary body) is a common ocular disorder that often does not respond to available treatments. Uveitis may be associated with an autoimmune condition (local or systemic), it may result from infection (e.g., bacterial; viral, such as herpes; fungal; or parasitic, such as toxoplasmosis), or it may be associated with other diseases, such as sarcoidosis or Behçet's disease. It can also develop following eye trauma or surgery. Uveitis can lead to vision loss by damage to the iris, lens, or retina, or by causing glaucoma. Approximately 10% of the blindness in the United States is due to uveitis. Treatment consists primarily of local and/or systemic administration of corticosteroids, immunosuppressive drugs, antimetabolites such as methotrexate, or anti-inflammatory monoclonal antibodies such as infliximab. Despite treatment, many cases of uveitis ultimately lead to vision loss.

It has now been discovered that gallium compounds, particularly gallium maltolate, administered to the eye topically or intraocularly, are highly effective at treating ocular diseases and disorders, including ocular inflammation (such as uveitis), infections, and cancers. The topical ocular or intraocular administration of gallium is novel, as are topical ocular and intraocular gallium compositions.

### SUMMARY OF THE INVENTION

Accordingly, it is a primary object of the invention to provide topical ocular and intraocular pharmaceutical compositions, methods, and drug delivery systems for treating diseases and disorders of the eye.

In a compositional embodiment of the invention, pharmaceutical gallium compositions are provided for topical ocular and intraocular administration.

In a methodological embodiment of the invention, methods are provided for using topical ocular and intraocular pharmaceutical gallium compositions to treat diseases and disorders of the eye.

In another methodological embodiment of the invention, methods are provided for treating an adverse condition of the eye or adnexal eye tissue in an individual afflicted with such a condition, wherein the adverse condition is not associated with infection by obligate intracellular prokaryotes or DNA viruses, comprising administering to the affected eye or eyes a therapeutically effective amount of a ophthalmically administrable pharmaceutical composition comprising a carrier suitable for ophthalmic administration and an active agent, wherein the active agent is a pharmaceutically acceptable gallium compound other than gallium deferoxamine.

Additional objects, advantages and novel features of the invention will be set forth in part in the description that follows, and in part will become apparent to those skilled in the art upon examination of the following, or may be learned by practice of the invention.

### DETAILED DESCRIPTION OF THE INVENTION

Before the present compositions, methods, and drug delivery systems of the invention are disclosed and described, it is to be understood that this invention is not limited to specific formulations, i.e., specific carrier materials or the like, to specific dosage regimens, or to specific drug delivery systems, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a gallium compound” includes mixtures of such compounds; reference to “a carrier” includes mixtures of two or more carriers; and the like.

The terms “topical ocular administration” and “topical ophthalmic administration” are used interchangeably herein, and are used in their conventional sense to mean delivery of a topical drug or pharmacologically active agent to the surface of the eye, including the conjunctiva and other adnexal tissues of the eye, such as the eyelids and tear ducts.

The term “intraocular administration” is used in its conventional sense to mean delivery of a drug or pharmacologically active agent to the interior of the eye, such as the aqueous humor, the vitreous humor, or other regions within the eye.

The term “patient” is meant to include a human or a veterinary patient. Within the context of the present invention, veterinary patients are intended to include both mammalian and non-mammalian veterinary patients, the latter including such veterinary patients as, for example, lizards and birds.

The terms “active agent,” “drug,” and “pharmacologically active agent” are used interchangeably herein to refer to a chemical material or compound that, when administered to an organism (human or animal) induces a desired pharmacologic effect, such as a reduction in inflammation or infection.

The terms “to treat” and “treatment” as used herein encompass the usual meanings of these terms plus the usual meanings of the terms “to prevent” and “prevention”. Thus, for example, “treatment” of uveitis, as the term “treatment” is used herein, encompasses both prevention of uveitis in a predisposed individual and treatment of uveitis in an individual who has such a disease.

By the term “effective” amount of a drug is meant a sufficient amount of a drug to provide the desired effect and performance at a reasonable benefit/risk ratio attending any medical treatment.

The term “vehicle” or “carrier” as used herein refers to a vehicle suitable for administration of a drug, and includes any such materials known in the art, e.g., any liquid or non-liquid carrier, gel, cream, ointment, lotion, paste, emulsifier, solvent, liquid diluent, powder, or the like, which is stable with respect to all components of the pharmaceutical formulation.

This invention includes topical ocular and intraocular pharmaceutical compositions suitable for the localized administration of gallium to the eye, and methods for using such compositions to treat eye diseases and disorders. Any gallium compositions suitable for topical ocular or intraocular administration are included in this invention.

Treatment is applicable to human and veterinary patients, including particularly mammals and birds. Mammalian veterinary subjects include, without limitation, dogs, cats, and members of the Equidae, Bovidae, Caprinae, and Suidae. Veterinary subjects also include, without limitation, reptiles, amphibians, and fish.

The topical ocular pharmaceutical compositions comprise a topical ocular carrier and an effective amount of a pharmaceutically acceptable gallium compound.

The intraocular pharmaceutical compositions comprise a carrier suitable for intraocular administration, such as, for example, Ringer's solution or balanced salt solution, and an effective amount of a pharmaceutically acceptable gallium compound. The carrier for intraocular pharmaceutical compositions is preferably free of preservatives and endotoxins.

The invention provides topical ocular formulations in the form of eye drops, eye washes, contact lens solutions, ointments, gels, patches, packs, depot formulations, slow release formulations, aerosols, and the like. In various embodiments, the topical ocular formulation may be provided in single or multi-dose containers or dispensers.

The invention also provides intraocular formulations in the form of injectable solutions, eye irrigating solutions, volume replacement solutions, gels, depot formulations, slow release formulations, and the like. In various embodiments, the intraocular formulation may be provided in single or multi-dose containers or dispensers, or in implantable intraocular devices.

Gallium compounds usable in this invention include, without limitation, gallium nitrate, gallium sulfate, gallium citrate, gallium chloride, gallium complexes of 3-hydroxy-4-pyrones including gallium maltolate, gallium tartrate, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexes of kenpaullone and its derivatives, and any other pharmaceutically acceptable gallium salts, organic salts, inorganic compounds, chelates, coordination compounds, and organometallic compounds. Gallium maltolate, tris(3-hydroxy-2-methyl-4H-pyran-4-onato)gallium, is a preferred gallium compound of the invention; this compound is described, for example, in U.S. Pat. No. 5,981,518. Without being restricted to any theory or hypothesis, gallium maltolate has a number of advantages, including that it is electrically and pH neutral in aqueous solution, so it is not irritating to the eye, and that it is significantly soluble in aqueous and lipidic solutions, so that it penetrates the cornea, skin, and cell membranes readily.

Preferred topical ocular formulations herein are colorless, odorless solutions, ointments, and gels. Particularly preferred are aqueous solutions and gels.

The topical ocular carrier, as noted above, is one that is generally suited to topical ocular drug administration and includes any such materials known in the art. The topical ocular carrier is selected so as to provide the composition in the desired form, e.g., as a liquid, paste, gel, or ointment, and may be comprised of a material of either naturally occurring or synthetic origin. It is essential that the selected carrier not adversely affect the active agent or other components of the topical ocular formulation. It is also essential that the selected carrier not be irritating, allergenic, or otherwise harmful to the eye or surrounding tissues of most subjects. Examples of suitable topical ocular carriers for use herein include water, glycerin, petroleum jelly, petrolatum, and the like.

The topical ocular carrier used for solutions or suspensions is preferably water, and less preferably lipidic or oily. Aqueous solutions are preferred, as they are easy to instill, do not interfere with vision, and rarely cause adverse reactions. Suspensions have the advantage of more extended action, but the disadvantage that they may contain a few particles that are large enough to cause irritation.

Topical ocular liquids of the invention, such as solutions or suspensions, are usually instilled into the eye as eye drops, which are applied to the surface of the eye, the conjunctiva, the conjunctival sac, or the space between the eye and the eyelid (the cul-de-sac). A single drop generally has a volume of approximately 30 μL.

The invention also provides topical ocular ointments. Eye ointments are sterile preparations for external application to the eye, generally to the conjunctival sac or lid margin. They may have advantages over solutions of more prolonged contact and effect, minimal irritation on initial installation, slower movement into lacrimal ducts, greater storage stability, and less likelihood of contamination problems. Their possible disadvantages are that they may produce a film over the eye thereby blurring vision, and may interfere with the firm attachment of new corneal epithelial cells to their normal base. Ointments affect the outside and edges of the eyelids, the conjunctiva, the cornea, and the iris, depending on their ability to penetrate the outer covering of the eyeball. Ophthalmic ointments usually contain a white petrolatum-mineral oil base, often including anhydrous lanolin, while some have a polyethylene-gelled mineral oil base. Whichever base is selected, it must be nonirritating to the eye, permit diffusion of the drug throughout the secretions bathing the eye, and retain the activity of the medicament for a reasonable period of time under proper storage conditions.

The practice of the present invention will employ, unless otherwise indicated, conventional techniques of drug formulation, particularly topical ocular and intraocular drug formulation, which are within the skill of the art. Such techniques are fully explained in the literature. See, for example, Remington: The Science and Practice of Pharmacy (Lippincott Williams & Wilkins, 2000), as well as Goodman & Gilman's The Pharmacological Basis of Therapeutics, 9th Ed. (New York: McGraw-Hill, 1996) and Ansel et al., Pharmaceutical Dosage Forms and Drug Delivery Systems, 6^{th} Ed. (Media, Pa.: Williams & Wilkins, 1995).

Formulations for topical ophthalmic administration can include ophthalmically acceptable excipients, such as tonicity-adjusting agents, pH-adjusting agents, buffering agents, preservatives, comfort enhancing agents, viscosity-modifying agents, stabilizing agents, and the like. For example, sodium chloride, glycerin, mannitol or the like may be used as an isotonic agent; p-hydroxybenzoic acid ester, benzalkonium chloride, or the like as a preservative; sodium hydrogen phosphate, sodium dihydrogen phosphate, boric acid, or the like as a buffering agent; sodium edetate or the like as a stabilizer; polyvinyl alcohol, polyvinyl pyrrolidone, polyacrylic acid or the like as a viscosity enhancing agent; and sodium hydroxide, hydrochloric acid, or the like as pH controllers. The pH is typically adjusted to between 4.5 and 8 for topical ocular use, most commonly between 6.5 and 7.5.

Other additives, known to those skilled in the art, may be included in the topical ocular formulations of the invention. For example, solvents may be used to solubilize certain drug substances. Other optional additives include permeation enhancers, anti-oxidants, gelling agents, thickening agents, and the like. The topical compositions of the invention may be also optionally comprise one or more other active agents, including, without limitation, analgesics, anesthetics, anti-inflammatory agents, anticancer agents, antiglaucoma agents, antibiotics, antimicrobial agents, antibacterial agents, antifungal agents, antiviral agents, antiparasitic agents, and antihelminthic agents.

Compositions of this invention may also be administered by other localized means to the eye. Ophthalmic packs may be used to give prolonged contact of the solution with the eye. For example, a cotton pledget saturated with an opthalmologically suitable solution of a compound of this invention may be inserted into the superior or inferior formix. Medicated controlled-release ophthalmic disks may produce effects both more intense and prolonged than solutions. See, e.g., U.S. Pat. No. 4,190,642, which discloses a controlled-release device for administering compounds to the eye, which may be useful in the practice of this invention.

The compounds of the invention may also be administered by the way of iontophoresis. This procedure keeps the solution in contact with the cornea in an eyecup bearing an electrode. Diffusion of the drug is effected by difference of electrical potential (see, for example, Remington's Pharmaceutical Sciences, 15th Ed., pp. 1489-1504, 1975).

In a preferred topical ocular formulation of the invention, the gallium compound is present in an amount such that the elemental gallium content is generally about 0.00001 to about 5 percent by weight of the formulation, preferably about 0.001 to about 2 percent, and most preferably about 0.05 to about 1.0 percent.

In a preferred intraocular formulation of the invention, the gallium compound is present in an amount such that the elemental gallium content is generally about 0.00001 to about 2 percent by weight of the formulation, preferably about 0.0001 to about 0.5 percent, and most preferably about 0.01 to about 0.1 percent.

The topical ocular compositions of the invention may be applied by any practical, medically acceptable means. For example, application may be made using droppers, patches, fingers, or other means.

The intraocular compositions of the invention may also be administered by any practical, medically acceptable means. Administration can be accomplished by intraocular injection, cannula, implanted intraocular device, or other invasive means designed to introduce precisely metered amounts of a desired formulation to a particular compartment or tissue within the eye (e.g., posterior chamber or retina). An intraocular injection, as examples, may be into the vitreous (intravitreal), under the conjunctiva (subconjunctival), behind the eye (retrobulbar), into the sclera, or under the Capsule of Tenon (sub-Tenon), and may be in a depot form. Intraocular injection is preferably through a self sealing 25 to 30 gauge needle or other suitably calibrated delivery device. Injection into the eye may be through the pars plana via the self-sealing needle. The intraocular compositions of the invention may also be used as intraocular irrigating solutions or volume replacement solutions. Other intraocular routes of administration and injection sites and forms are also contemplated and are within the scope of the invention.

In one intraocular embodiment, the formulation is intraocularly injected to treat or prevent an ophthalmic condition. When administering the formulation by intraocular injection, the active agents should be concentrated to minimize the volume for injection. Preferably, the volume for injection is less than about 5 mL. Volumes such as this may require compensatory drainage of ocular fluid to prevent increases in intraocular pressure and leakage of the injected fluid through the opening formed by the delivery needle. More preferably, the volume injected is between about 1.0 mL and 0.01 mL. Most preferably, the volume for injection is approximately 0.1 mL.

Intraocular injection may be achieved by a variety of methods well known in the art. For example, the eye may be washed with a sterilizing agent such as Betadine® and the intraocular gallium formulation is injected in an appropriate carrier with a fine gauge needle (e.g., 27 gauge). It may be necessary to prepare the eye for injection by application of positive pressure prior to injection. In some cases, paracentesis may be necessary. Local anesthetic or general anesthetic may be necessary.

It will be recognized by those skilled in the art that the optimal quantity and spacing of individual dosages of the topical ocular or intraocular gallium compositions of the invention will be determined by the nature and extent of the condition being treated, the form, route and site of administration, and the particular individual undergoing treatment, and that such optimums can be determined by conventional techniques. It will also be appreciated by one skilled in the art that the optimal dosing regimen, i.e., the number of doses of a gallium composition of the invention, can be ascertained using conventional course of treatment determination tests.

For the topical ocular formulations of the invention, a dosing regimen will generally involve administration at least once weekly to the affected eye or eyes, and preferably one to four times daily, until the inflammation, infection, or other symptoms have subsided. A preferred method of administration is by eye drops, wherein one to two drops of solution are typically administered in one dose. Typical topical ocular doses per eye of the gallium composition, expressed as the amount of contained elemental gallium, are, for example, about 0.00005 to 2 mg, preferably about 0.001 to 0.5 mg, and more preferably about 0.01 to 0.1 mg; such doses are typically administered, for example, once per week to six times per day, and more typically one to four times per day.

When administered intraocularly, typical doses per eye, expressed as the amount of contained elemental gallium, are, for example, about 0.00001 to 1 mg, preferably about 0.0001 to 0.2 mg, and more preferably about 0.001 to 0.05 mg.

When administered continuously, as by the use of a ophthalmic pack, patch, intraocular device, depot formulation, intraocular drug reservoir, or pump, typical daily doses of the gallium composition, expressed as the amount of contained elemental gallium, per eye are, for example, about 0.0001 to 5 mg, preferably about 0.001 to 1 mg, and more preferably about 0.01 to 0.5 mg.

Adverse conditions of the eye and adnexal eye tissue treatable by the gallium compositions of this invention include, without limitation, inflammation, including intraocular inflammation such as uveitis (including autoimmune uveitis; infectious uveitis; and uveitis associated with acute posterior multifocal placoid pigment epitheliopathy, ankylosing spondylitis, Behçet's disease, birdshot retinochoroidopathy, brucellosis, Herpes simplex, Herpes zoster, inflammatory bowel disease, juvenile rheumatoid arthritis, Kawasaki's disease, leptospirosis, Lyme disease, multiple sclerosis, presumed ocular histoplasmosis syndrome, psoriasis, psoriatic arthritis, radiation damage, Reiter's syndrome, sarcoidosis, surgery, syphilis, systemic lupus erythematosus, toxocariasis, toxoplasmosis, trauma, tuberculosis, Vogt-Koyanagi-Harada syndrome, or other pathologic conditions); conjunctivitis; infection, including bacterial infection, viral infection, parasitic infection, protozoal infection, helminthic infection, and fungal infection; cancer of the eye or surrounding (adnexal) tissues, including intraocular melanoma, primary intraocular lymphoma, retinoblastoma, medulloepithelioma, choroidal melanoma, choroidal osteoma, ciliary body melanoma, iris melanoma, conjunctival Kaposi's sarcoma, lymphoma of the conjunctiva, melanoma and PAM with atypia, pigmented conjunctival tumors, squamous carcinoma and intraepithelial neoplasia of the conjunctiva, adnexal cancers (as of the eyelid or tear ducts, including squamous cell carcinoma, basal cell carcinoma, sebaceous carcinoma, and malignant melanoma), and cancers metastatic to the eye or adnexal tissues; choroidal hemangioma; choroidal nevus; choristoma; orbital dermoid cysts; nevus of Ota; pingueculum; pterygium; glaucoma; macular degeneration; retinopathy, including diabetic retinopathy; blepharitis; styes; and chalazions.

In a preferred embodiment of the invention, treatable adverse conditions of the eye and adnexal eye tissue are those that are not associated with infection by obligate intracellular prokaryotes or DNA viruses. Such treatable eye diseases and disorders include, without limitation, inflammation, including intraocular inflammation such as uveitis (including autoimmune uveitis; and uveitis associated with acute posterior multifocal placoid pigment epitheliopathy, ankylosing spondylitis, Behçet's disease, birdshot retinochoroidopathy, brucellosis, inflammatory bowel disease, juvenile rheumatoid arthritis, Kawasaki's disease, leptospirosis, Lyme disease, multiple sclerosis, presumed ocular histoplasmosis syndrome, psoriasis, psoriatic arthritis, radiation damage, Reiter's syndrome, sarcoidosis, surgery, syphilis, systemic lupus erythematosus, toxocariasis, toxoplasmosis, trauma, Vogt-Koyanagi-Harada syndrome, or other pathologic conditions); conjunctivitis; certain infections, including infections with bacteria that are not obligate intracellular pathogens, infections with non-DNA viruses, parasitic infections, protozoal infections, helminthic infections, and fungal infections; cancer of the eye or surrounding (adnexal) tissues, including intraocular melanoma, primary intraocular lymphoma, retinoblastoma, medulloepithelioma, choroidal melanoma, choroidal osteoma, ciliary body melanoma, iris melanoma, conjunctival Kaposi's sarcoma, lymphoma of the conjunctiva, melanoma and PAM with atypia, pigmented conjunctival tumors, squamous carcinoma and intraepithelial neoplasia of the conjunctiva, adnexal cancers (as of the eyelid or tear ducts, including squamous cell carcinoma, basal cell carcinoma, sebaceous carcinoma, and malignant melanoma), and cancers metastatic to the eye or adnexal tissues; choroidal hemangioma; choroidal nevus; choristoma; orbital dermoid cysts; nevus of Ota; pingueculum; pterygium; glaucoma; macular degeneration; retinopathy, including diabetic retinopathy; blepharitis; styes; and chalazions. Bacterial infections included in this preferred embodiment are, without limitation, infections by Gram positive bacteria, such as Staphylococcus aureus, Staphylococcus aureus drug resistant, Staphylococcus epidermidis, and other Staphylococcus species and strains; Streptococcus pyogenes Streptococcus pneumoniae; and other Streptococcus species and strains; Enterococcus faecalis and other Enterococcus species and strains; Actinomyces spp.; Propionibacterium spp.; and Gram negative bacteria, such as Escherichia coli, Pseudomonas aeruginosa, Proteus mirabilis, Enterobacter aerogenes, Haemophilus influenzae, Haemophilus spp., Pasteurella spp., Neisseria gonorrhoeae, and Klebsiella spp. Fungal infections included in this preferred embodiment include, without limitation, infections by Fusarium, Aspergillus, Curvularia, Paecilomyces, or Candida.

In another preferred embodiment, the gallium compound used in the invention is not a gallium deferoxamine complex.

All patents, patent documents, and publications cited herein are hereby incorporated by reference in their entirety for their disclosure concerning any pertinent information not explicitly included herein.

It is to be understood that while the invention has been described in conjunction with the preferred specific embodiments thereof, the foregoing description, as well as the examples that follow, are intended to illustrate and not limit the scope of the invention. Other aspects, advantages, and modifications will be apparent to those skilled in the art to which the invention pertains.

### EXPERIMENTAL

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### Example 1

### Topical Ocular Solution of Gallium Maltolate

An aqueous gallium maltolate solution for topical application to the eye was prepared at room temperature with the following composition:

| | |
|---|---|
| Gallium maltolate | 0.5 w/v% |
| Dibasic Sodium Phosphate (anhydrous), USP | 0.5 w/v% |
| Sodium Chloride, USP | 0.65 w/v% |
| Benzalkonium Chloride | 0.01 w/v% |
| Sodium Hydroxide, NF | q.s. pH = 7.0 |
| Hydrochloric Acid, NF | q.s. pH = 7.0 |
| Purified Water | q.s. 100 v% |

### Example 2

### Topical Ocular Gallium Maltolate Gel

A gallium maltolate gel formulation for topical application to the eye was prepared at room temperature with the following composition:

| | |
|---|---|
| Gallium maltolate | 0.25 w/v% |
| Carbopol 974 P | 0.8 w/v% |
| Edetate Disodium | 0.01 w/v% |
| Polysorbate 80 | 0.05 w/v% |
| Benzalkonium Chloride | 0.01 w/v% |
| Sodium Hydroxide, NF | q.s. pH = 7.2 |
| Hydrochloric Acid, NF | q.s. pH = 7.2 |
| Purified Water | q.s. 100 v% |

### Example 3

### Gallium Maltolate Solution for Intraocular Administration

A gallium maltolate solution for intraocular administration, using a balanced salt solution, was prepared at room temperature with the following composition:

| | | |
|---|---|---|
| Gallium maltolate | 0.1 | w/v% |
| Sodium chloride | 0.64 | w/v% |
| Potassium chloride | 0.075 | w/v% |
| Calcium chloride dihydrate | 0.048 | w/v% |
| Magnesium chloride hexahydrate | 0.03 | w/v% |
| Sodium acetate trihydrate | 0.39 | w/v% |
| Sodium citrate dihydrate | 0.17 | w/v% |

| | |
|---|---|
| Sodium Hydroxide, NF: | q.s. pH = 7.0 |
| Hydrochloric Acid, NF | q.s. pH = 7.0 |
| Purified Water for Injection | q.s. 100 v% |

### Example 4

### Use of Topical Ocular Gallium Maltolate Solution to Treat Uveitis

The topical ocular gallium maltolate formulation of Example 1 is used to treat autoimmune uveitis in the right eye of a 55 year old woman. A drop (about 30 μL, containing about 0.15 mg of gallium maltolate) of the formulation is instilled in the affected eye four times per day, at about 8 AM, 12 PM, 4 PM, and 8 PM, for seven days. After one day of treatment, the inflammation associated with the uveitis has noticeably subsided, and after seven days of treatment, the uveitis has entirely resolved.

I claim:

1. A method for treating an adverse condition of the eye or adnexal eye tissue in an individual afflicted with such a condition, wherein the adverse condition is not associated with infection by obligate intracellular prokaryotes or DNA viruses, comprising administering to the affected eye or eyes a therapeutically effective amount of a ophthalmically administrable pharmaceutical composition comprising a carrier suitable for ophthalmic administration and an active agent, wherein the active agent is a pharmaceutically acceptable gallium compound other than gallium deferoxamine.

2. The method of claim 1, wherein the adverse condition is an inflammation.

3. The method of claim 2, wherein the adverse condition is uveitis.

4. The method of claim 1, wherein the adverse condition is an infection.

5. The method of claim 4, wherein the infection is bacterial.

6. The method of claim 4, wherein the infection is viral.

7. The method of claim 4, wherein the infection is fungal.

8. The method of claim 4, wherein the infection is parasitic, protozoal, or helminthic.

9. The method of claim 1, wherein the adverse condition is cancer.

10. The method of claim 1, wherein the gallium compound is selected from the group comprising gallium nitrate, gallium sulfate, gallium citrate, gallium chloride, gallium complexes of 3-hydroxy-4-pyrones, gallium maltolate, gallium tartrate, gallium succinate, gallium gluconate, gallium palmitate, gallium 8-quinolinolate, gallium porphyrins, and gallium pyridoxal isonicotinoyl hydrazone.

11. The method of claim 10, wherein the gallium compound is a gallium complex of a 3-hydroxy-4-pyrone.

12. The method of claim 11, wherein the gallium complex of a 3-hydroxy-4-pyrone is gallium maltolate.

13. The method of claim 10, wherein the gallium compound is gallium nitrate.

14. The method of claim 1, wherein the pharmaceutical composition is in the form of a liquid.

15. The method of claim 1, wherein the pharmaceutical composition is in the form of a gel.

16. The method of claim 1, wherein the pharmaceutical composition is in the form of an ointment.

17. The method of claim 1, wherein the pharmaceutical composition is administered topically to the eye, and the pharmaceutical composition comprises a carrier suitable for topical ocular administration.

18. The method of claim 1, wherein the pharmaceutical composition is administered topically to the adnexal tissue of the eye, and the pharmaceutical composition comprises a carrier suitable for topical ocular administration.

19. The method of claim 1, wherein the pharmaceutical composition is administered intraocularly, and the pharmaceutical composition comprises a carrier suitable for intraocular administration.

20. The method of claim 1, wherein the pharmaceutical composition is present in a drug reservoir contained within the eye.

21. The method of claim 1, wherein the elemental gallium content represents about 0.00001 to about 5 percent by weight of the pharmaceutical composition.

22. The method of claim 1, wherein the elemental gallium content represents about 0.001 to about 2 percent by weight of the pharmaceutical composition.

23. The method of claim 1, wherein the elemental gallium content represents about 0.05 to about 1.0 percent by weight of the pharmaceutical composition.

24. The method of claim 1, wherein the elemental gallium content represents about 0.01 to about 0.1 percent by weight of the pharmaceutical composition.

25. The method of claim 12, wherein the gallium maltolate content represents about 0.0001 to 5 percent by weight of the pharmaceutical composition.

26. The method of claim 12, wherein the gallium maltolate content represents about 0.01 to 1 percent by weight of the pharmaceutical composition.

27. The method of claim 1, wherein the pharmaceutical composition comprises one or more additional active agents.

28. The method of claim 1, wherein the individual is human.

29. The method of claim 1, wherein the individual is a veterinary subject.
